(19) United States
(12) Patent Application Publication
(10) Pub. No.: US 2025/0198887 A1
(43) Pub. Date: Jun. 19, 2025

(54) DEVICE FOR SHAKING SAMPLES

(71) Applicant: Infors AG, Bottmingen (CH)

(72) Inventors: Alexander HAWRYLENKO, Bottmingen (CH); Johan DOPPLER, Lutter (FR)

(21) Appl. No.: 18/843,559

(22) PCT Filed: Mar. 3, 2023
(86) PCT No.: PCT/EP2023/055468

§ 371 Date: Sep. 3, 2024

(30) Foreign Application Priority Data
Mar. 4, 2022 (WO) PCT/EP2022/055636

(51) Int. Cl. G01N 1/38

(52) U.S. Cl. CPC G01N 1/38; G01N 2001/382

(57) ABSTRACT

A device for shaking samples includes a carrier (62), a drive element (66) which is rotatably bearing-mounted on the carrier (62), and a tray (61) arranged for loading with the samples (69). Furthermore, the device includes a tray shaft (67), which is connected to the tray (61) and is bearing-mounted eccentrically on the drive element (66), and an openable housing (64). The tray (61), the tray shaft (67), the drive element (66) and the carrier (62) are located in an interior space within the housing (64). In addition, the device includes a main drive shaft (63) drivable by a drive (65) and which extends orthogonally to the tray (61) at least partially through the housing (64). The drive element (66) is drivable via the main drive shaft (63). The carrier (62) together with the tray (61) is pivotably bearing-mounted about the main drive shaft (63), wherein the carrier (62) together with the tray (61) is at least partially pivotable out of the housing (64) in the open state.

### CROSS-REFERENCE TO RELATED APPLICATIONS

This application is a National Stage application of International Patent Application No. PCT/EP2023/055468, filed on Mar. 3, 2023, which claims priority to International Patent Application No. PCT/EP2022/055636, filed on Mar. 4, 2022, each of which is hereby incorporated by reference in its entirety.

### TECHNICAL FIELD

The invention relates to a device for shaking samples, in particular a laboratory shaker, in particular for shaking and/or mixing samples containing liquid.

### BACKGROUND

Shakers are used to shake and/or mix liquids, e.g. cell cultures, biofuels or blood samples, in vessels. The shaken unit often comprises a tray on which the vessels, e.g. Erlenmeyer flasks, test tubes or other ampoules, containing the samples are placed. On the one hand, a high shaking frequency is desirable for good mixing. On the other hand, liquid can be spilled or splashed during the shaking process, whereby other components such as a housing around the tray and/or a drive of the shaker can also be contaminated. With conventional shakers, the trays can only be removed with considerable effort. This means that cleaning after contamination is also a considerable effort, especially as some components are difficult to access due to the housing. The poor accessibility of the trays and samples also makes automation more difficult with conventional shakers, e.g. manipulation of the samples using a robot arm.

The task is therefore to provide a device for shaking samples that is easy to operate, whereby the trays are easily accessible and thus, in particular, the cleaning effort in the event of contamination is as low as possible and/or automated manipulation of the samples is possible.

### SUMMARY

This problem is solved by a device for shaking samples according to claim 1. By a sample is meant in particular a substance or a composition of substances containing a liquid, e.g. a cell culture, a biofuel or a blood sample. The sample is usually contained in a vessel, e.g. a test tube or a microtiter plate, which in turn can be held by a vessel holder or stand. The device comprises
a carrier: The carrier is especially designed to carry or support a tray;
a drive element that is rotatably bearing-mounted on the carrier: The drive element can, for example, comprise a hollow shaft or a drive pulley;
a tray for loading the samples: In particular, the tray is a planar element on which the samples can be mounted. Advantageously, the tray comprises fastening elements for fastening at least one vessel with a sample or for fastening at least one vessel holder or stand;
a tray shaft which is connected, in particular firmly, to the tray and is bearing-mounted eccentrically on the drive element: Advantageously, the tray shaft is attached centrally to the tray, in particular close to a center of gravity of the tray or of the tray with an intended load of samples. The term “close” refers in particular to an area around the center of gravity of up to +/−10% of the length and width of the tray. Due to the central attachment, the tray shaft (with the intended load) supports the tray close to the center of gravity;

The eccentricity (of the bearing) of the tray shaft on the drive element is the distance between an axis of rotation of the tray shaft and an axis of rotation of the drive element, which in particular run parallel to each other. The eccentricity determines the deflection of the tray (and therefore the samples) during shaking. In particular, the eccentricity of the bearing of the tray shaft on the drive element can be between 0.5 and 50 mm, in particular between 1 and 3 mm. This leads to a deflection of the tray which is adapted for shaking samples in smaller vessels, e.g. test tubes or microtiter plates;
an openable housing, whereby the tray, the tray shaft, the drive element and the carrier are located in an interior space within the housing: On the one hand, the housing can serve to close off the tray and samples from an environment of the device. On the other hand, the device can comprise a climate control element that is set up to control the temperature and/or humidity in an interior of the housing. For this purpose, and in particular to create ideal environmental conditions for the samples, the climate control element may comprise, for example, a heater, a cooler, a humidifier and/or a dehumidifier, which may be attached to the housing. To openably close the housing and thus maintain the ideal ambient conditions, the housing advantageously comprises a door, e.g. a hinged door, in particular on a front side of the housing;
a main drive shaft which can be driven by a drive, e.g. comprising a motor, and which extends orthogonally to the tray at least partially through the housing: Advantageously, the main drive shaft extends vertically through the housing, i.e. in particular orthogonally to a bearing surface of the housing, which may be provided, for example, by a plane through feet on the housing. The tray then runs horizontally in the housing, i.e. in particular orthogonally to gravity when used as intended. It is possible, but not necessary, for the main drive shaft to run through the tray or through a plane in the extension of the tray.

The drive element can be driven via the main drive shaft. This means that the tray can be shaken by a drive, e.g. with a motor, via the main drive shaft and the drive element.

In addition, the carrier together with the tray is pivotably bearing-mounted around the main drive shaft. The carrier can be mounted via a bearing, in particular on the main drive shaft itself or on the housing or a carrier element attached to the housing. The pivotable bearing provides better accessibility to the tray, e.g. for loading and unloading the tray with samples. In particular, this makes it possible to automate the operation of the shaker, e.g. via a computer-controlled robot arm. At the same time, better accessibility to the interior of the housing is achieved, for example by pivoting the tray out of the housing. This, in turn, makes it easier to clean the interior and the components inside and thus to work under controlled, in particular sterile, conditions.

### Pivotable Tray

The carrier together with the tray can be pivoted at least partially, in particular more than 50% or more than 70% of a surface of the tray, out of the housing in the open state. In one embodiment, the carrier together with the tray can be pivoted by at least 45°, in particular at least 90°, around the main drive shaft. These measures in turn improve the accessibility of the tray and the interior of the housing.

Advantageously, the tray comprises an opening through which the main drive shaft passes. This enables a compact design of the device, in particular with several trays driven via the main drive shaft. Furthermore, it is advantageous that the opening is located in an edge region of the tray, in particular less than 20% of a length and/or width of the tray away from an edge of the tray. Due to such a position of the opening or the main drive shaft relative to the tray, the tray can be pivoted far out of the housing, i.e. in particular more than 50% or more than 70% of the surface of the tray. It is particularly suitable for the opening to be located in a corner area of the tray, in particular less than 20% of the length and width of the tray away from the edge of the tray. Preferably, the opening or the main drive shaft is located in the corner area of the tray close to the door.

Alternatively, the main drive shaft can also run outside the surface of the tray. In this case, a compact design of the housing can be achieved by running the main drive shaft close to the tray, in particular less than 20% of the length or width of the tray away from the edge of the tray. Preferably, the main drive shaft runs close to a corner of the tray near the door.

In one embodiment, the tray has a rectangular shape. By “rectangular” is meant any substantially rectangular shape, e.g. with rounded corners or a parallelogram or trapezoid. In particular, the tray can have a length of between 50 and 100 cm and/or a width of between 30 and 70 cm, which makes it possible to load a large number of samples and to use conventional vessels and vessel holders.

Furthermore, the carrier comprises a latch at the end remote from the main drive shaft for releasable attachment of the carrier to the housing or to a support structure in the housing. This enables better fastening of the carrier, which supports the tray, during operation. In particular, the latch is designed to prevent the carrier (and thus also the tray) from pivoting around the main drive shaft. At the same time, the latch, which can include a plug-in lock or a latchable flap, for example, enables the tray to be unlocked and swung out of the housing quickly and in a user-friendly manner.

In addition, the device can comprise a belt that is set up to drive the drive element via the main drive shaft. In particular, in this case a first pulley is attached to the drive element and a second pulley is attached to the main drive shaft, over which the belt runs. Alternatively, a gear drive for driving the drive element via the main drive shaft is also conceivable.

Advantageously, the tray is secured against rotation relative to the carrier, in particular so that it does not rotate or at least does not rotate significantly, in particular not by more than 10°, when the drive element rotates. Several variants are conceivable for securing the tray against rotation in this way: On the one hand, a mechanical guide that is attached to the housing and is set up to restrict the degrees of freedom of the tray to translations in the tray plane, e.g. via elastic elements such as springs, which act in particular in the edge area of the tray. On the other hand, the drive itself, e.g. via the belt, can be designed to secure the tray against rotation. For this purpose, the device can comprise a second tray shaft which is connected to the tray and is bearing-mounted eccentrically, in particular with the same eccentricity as the tray shaft, on the main drive shaft, e.g. on the second pulley. The double bearing of the tray also prevents rotation.

In a particularly advantageous embodiment with first and second pulleys, first and second tray shafts and first and second drive elements, the tray comprises a first tray part and a second tray part. The first and second tray shafts are connected, in particular firmly, to the first and second tray parts and are bearing-mounted eccentrically on the first and second drive elements. To prevent rotation of the tray, the first and second tray parts are connected to each other by a linear guide. Such a linear guide is designed in particular to ensure that the first and second tray parts can move relative to each other along an axis of the linear guide, but cannot rotate relative to each other. In other words, the linear guide restricts all degrees of freedom of the tray parts in relation to each other with the exception of a degree of freedom of translation along the axis of the linear guide. The embodiment with two tray parts connected to a linear guide has the advantage that damaging forces on the bearing of the tray shafts, which are caused for example by thermal expansion of a one-piece tray, are prevented. This in turn enables higher shaking frequencies, a longer operating time and smoother running of the shaker.

In a further advantageous embodiment, the device also comprises a carrier element attached to the housing. The carrier together with the tray is pivotably bearing-mounted on the carrier element via a bearing, in particular via a plain bearing. Advantageously, a pivot axis of the carrier coincides with the main drive shaft. In particular, the carrier is therefore not in mechanical contact with the main drive shaft. This mechanical separation of the main drive shaft and the bearing of the carrier reduces undesirable friction, wear and vibrations, especially at high shaking frequencies (and thus high rotation frequencies of the main drive shaft).

### Several Trays

In one embodiment, the device comprises at least one further tray together with a further tray shaft, a further drive element and a further carrier in the interior of the housing. In particular, the device can comprise at least five further trays, together with further tray shafts, further drive elements and further carriers in the interior of the housing. This increases the capacity of the device, i.e. in particular the number of samples that can be shaken simultaneously. Advantageously, the at least one further drive element or the at least five further drive elements can also be driven via the main drive shaft. In this way, a compact design with easy maintenance is achieved.

Advantageously, the at least one further tray is pivotably bearing-mounted around the main drive shaft independently of the other tray or trays. In particular, all trays can be pivoted independently of one another around the main drive shaft, e.g. out of the housing. This in turn improves the accessibility of the trays, e.g. when loading and unloading samples.

In order to avoid an imbalance in the case of several trays, it is advantageous that an angular position of a bearing of the additional tray shaft on the additional drive element deviates from an angular position of the bearing of the tray shaft on the drive element. Otherwise, especially when the trays are heavily loaded, an imbalance could act on the main drive shaft and cause the entire device to vibrate.

This can be avoided in particular if the angular position of the bearings of the various tray shafts differ from each other by 360°/N, where N is the number of trays in the device. This compensates for any imbalance on the main drive shaft.

### Drive

In one embodiment, the drive comprises a motor, e.g. an electric motor, which is mounted outside the housing. In particular, the motor is coupled to the main drive shaft via a gearbox. Mounting the motor outside the housing has the advantage that heat generated during operation of the motor is not introduced into the interior of the housing. For many applications or samples, temperature and/or humidity control, e.g. climate control as described above, is desirable. In particular, the humidity is kept close to the dew point, especially at a relative humidity of between 80% and 100%. However, if the interior needs to be cooled at the same time, e.g. due to the heat input from an engine in the interior, the humidity locally exceeds 100% at the climate control or radiator and condenses out. Condensation is undesirable because it can lead to an uncontrolled proliferation of foreign germs, which can be harmful to the samples. In particular, the interior of the housing should be thermally decoupled from the motor. This problem is solved by a motor mounted outside the housing.

In particular, the motor can be attached to the underside of the housing. This lowers the center of gravity of the device, i.e. closer to the bearing surface, and thus increases the stability of the device, especially at high shaking frequencies. The main drive shaft is advantageously guided through an opening in the underside of the housing.

In general, it is advantageous that the shaking frequency of the tray as a result of the drive is at least 1000 rpm, in particular at least 1500 rpm, at least 2000 rpm or at least 2500 rpm, e.g. at 3 mm diameter of the circular movement. Such a high shaking frequency is particularly suitable for shaking and mixing samples in small vessels, e.g. in test tubes or in microtiter plates. Similar forces on the samples are generated, for example, at a shaking frequency of 350 rpm and a diameter of the circular motion of 50 mm. In particular, high shaking frequencies of at least 1000 rpm, at least 1500 rpm, at least 2000 rpm or at least 2500 rpm lead to better mixing of the shaken samples as well as to faster oxygen transfer from the gas phase to the liquid phase, which enables good growth of cell cultures in the samples.

### Interior of the Housing

To further simplify cleaning of the interior and create a controlled environment for the samples, the device can be designed in accordance with the specifications for “Hygienic Design”, as specified, for example, in the ISO 14159 standard “Safety of machinery-Hygiene requirements for the design of machinery” or in various articles of FDA CFR 177.

In particular, the device can have the following advantageous features:
A part of the housing facing the interior can be at least 50%, in particular at least 75%, covered with stainless steel.
The part of the housing facing the interior can comprise rounded corners and edges. Advantageously, a radius of the corners and edges is at least 10 mm, in particular at least 15 mm.
At least a part, in particular at least 70%, of an underside of the inner chamber can run at an angle to the bearing surface of the housing. As a result, splashed or spilled sample material collects in the deepest area of the underside, in particular the area closest to the bearing surface, and can be easily removed from there. Advantageously, the angle between the underside of the inner chamber and the bearing surface of the housing is between 1° and 30°, in particular between 5° and 15°.
In addition, it is advantageous that the housing comprises an outlet opening, in particular through the housing, in the area of the underside of the interior closest to the bearing surface. The outlet opening can also be closable with regard to possible climate control.

Each of these features, alone or in combination with the other features, improves the cleanability of the interior and the components inside.

The embodiments and features described are to be regarded as disclosed in any combination as far as reasonably practicable. Especially with regard to easy handling and good cleanability, they have synergistic effects and advantages.

### BRIEF DESCRIPTION OF THE DRAWINGS

Further embodiments, advantages and applications of the invention are apparent from the dependent claims and from the following description with reference to the figures. Showing:

FIG. 1a is a perspective view of a device for shaking samples according to one embodiment of the invention;

FIG. 1b is a top view of the device for shaking samples according to a further embodiment of the invention;

FIG. 1c is a perspective view of a device for shaking samples according to a further embodiment of the invention;

FIG. 2 is a schematic section through a device for shaking samples with an internal motor according to the state of the art;

FIGS. 3a and 3b are schematic sections through a device for shaking samples with an external motor according to embodiments of the invention;

FIG. 4a is a top view of an embodiment of the device according to the invention;

FIG. 4b is a schematic section along the line AA′ in FIG. 4a;

FIG. 4c is a detailed view of the corner area B of FIG. 4b;

FIG. 5 is a schematic side view of an embodiment of the device according to the invention with several trays;

FIG. 6a is a schematic vertical section through a device for shaking samples according to one embodiment;

FIG. 6b is a detailed view of region C of FIG. 6a;

FIG. 6c is a schematic vertical section through a device for shaking samples according to a further embodiment;

FIG. 7 is a horizontal section or top view of a tray and a counterweight according to an embodiment of the invention;

FIGS. 8a, 8b and 8c are schematic drawings of a liquid sample in a vessel with increasing shaking frequencies;

FIG. 9 is a schematic section through a bearing with which the tray is bearing-mounted on the main drive shaft according to one embodiment;

FIG. 10a is a perspective view of a pivoting tray with a latching mechanism, according to one embodiment;

FIG. 10b is an enlargement of a pivoting tray with a latching mechanism as shown in FIG. 10a;

FIG. 11a is an embodiment with a split tray in a perspective view;

FIG. 11b is an embodiment with a split tray in a schematic horizontal section; and

FIG. 11c is an embodiment with a split tray in a schematic vertical section.

### DETAILED DESCRIPTION

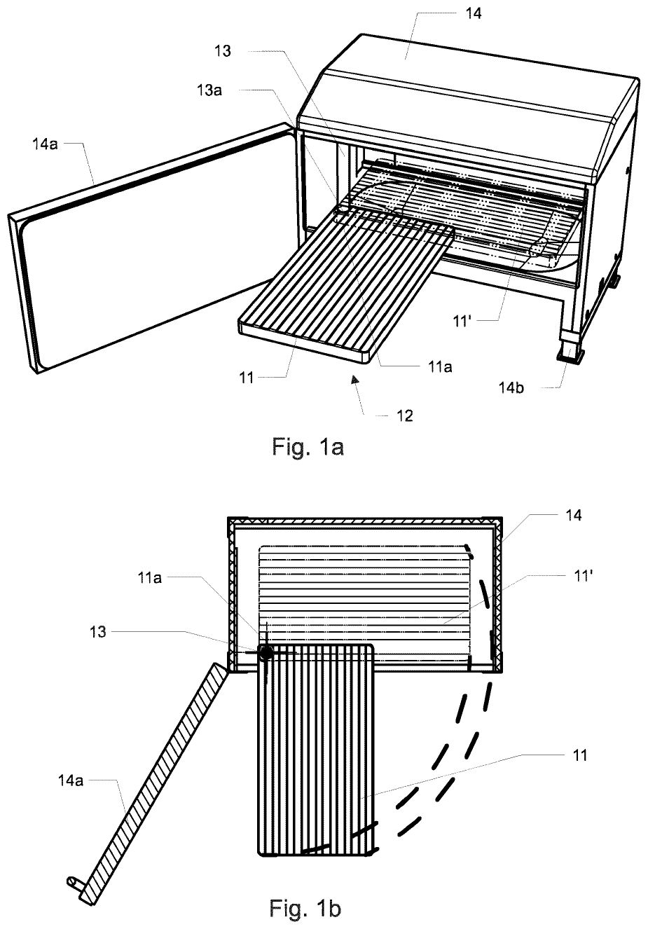

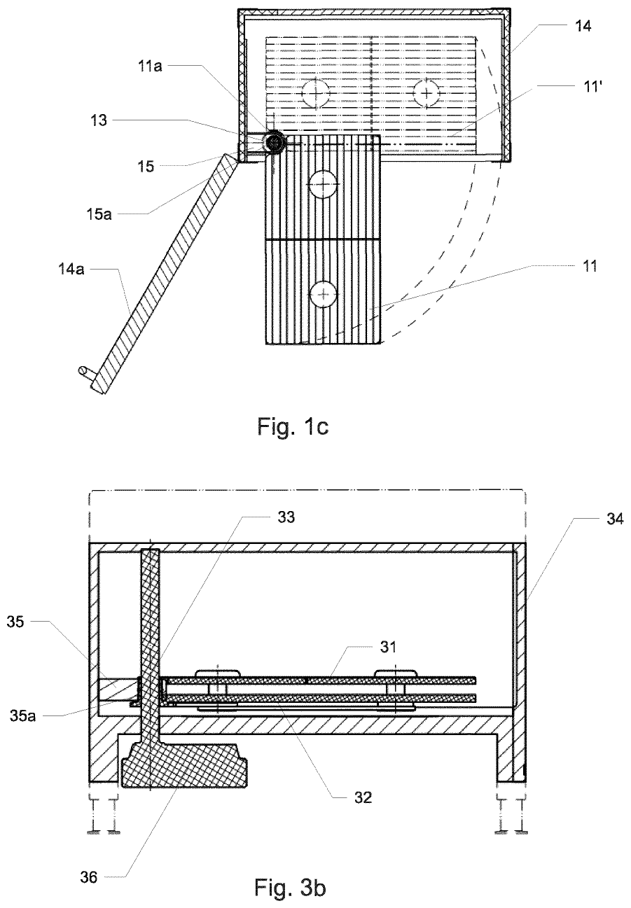

FIGS. 1a and 1b show a device for shaking samples, a so-called shaker, according to one embodiment. FIG. 1a is a perspective view of the device, while FIG. 1b shows a top view. The device comprises a tray 11 that can be loaded with samples. The tray 11 is mounted on a carrier 12 (not visible in FIGS. 1a and 1b) and is supported by it. The carrier 12 and with it the tray 11 can be pivoted about a main drive shaft 13. For this purpose, the carrier 12 is connected to the main drive shaft via a bearing 13a. In addition, the tray 11 has an opening 11a at its edge area, through which the main drive shaft 13 passes. The opening 11a is larger than a diameter of the main drive shaft 13, depending on the eccentricity of the bearing of the tray, in particular on the deflection of the shaking movement.

The tray 11 advantageously comprises an easy-to-clean surface, e.g. made of metal, at least on its upper side, i.e. the side facing the samples. This enables sterile operation of the device. Furthermore, the tray 11 can have a standard size of 850 mm×470 mm.

The tray 11 in the folded state 11′ and at least part of the main drive shaft 13 are enclosed by a housing 14, which comprises a door 14a for opening and closing. The housing 14 generally fulfills several functions: Firstly, it forms a stationary frame which can be placed, for example via feet 14b, on a table, in a laboratory or generally on a base. The shaking movement of the tray takes place relative to this stationary frame. Secondly, the housing provides protection of the environment of the device, e.g. from splashing or spilling of samples or from vapors, which is particularly desirable for harmful samples or in a sterile laboratory. Thirdly, controlled conditions, e.g. in terms of temperature and/or humidity, can be set in an interior of the housing, as is advantageous for many samples. For this purpose, the device may comprise a climate control for the interior (not shown in FIGS. 1a and 1b).

In FIGS. 1a and 1b, the tray is shown in two positions: on the one hand (marked 11) swung out of the housing 14, and on the other hand (dashed, marked 11′) swung into the housing in an operational state. In the figure, the angle between the two positions is 90°. In general, however, an angle of at least 45° is already advantageous, as it improves the accessibility of the tray 11 and the interior of the housing 14.

Furthermore, it can be seen in FIGS. 1a and 1b that an attachment of the main drive shaft 13 in the edge area or even corner area of the tray (as defined above) is advantageous for the pivotability and accessibility of the tray. Alternatively, the same advantage can be achieved by the main drive shaft 13 extending outside the tray 11 near its edge area (not shown in FIGS. 1a and 1b). In this case, the opening 11a in the tray 11 is superfluous and the carrier 12 projects horizontally beyond the tray 11. In general, the pivotability of the tray out of the housing improves the accessibility of the samples. In particular, a pivoting tray enables automation of the sample filling and removal process, as a robot arm, for example, can operate the device more easily under computer control.

The aforementioned advantages are also achieved by the modified mounting of the carrier 12 together with the tray 11 as in FIG. 1c. In contrast to the arrangement in FIG. 1a, the carrier 12 is bearing-mounted here via a bearing 15a, in particular a plain bearing, on a carrier element 15, which can be part of the housing 14 or attached to the housing 14. This mechanically decouples the carrier 12 from the main drive shaft 13. This results in smoother running and less wear on the device.

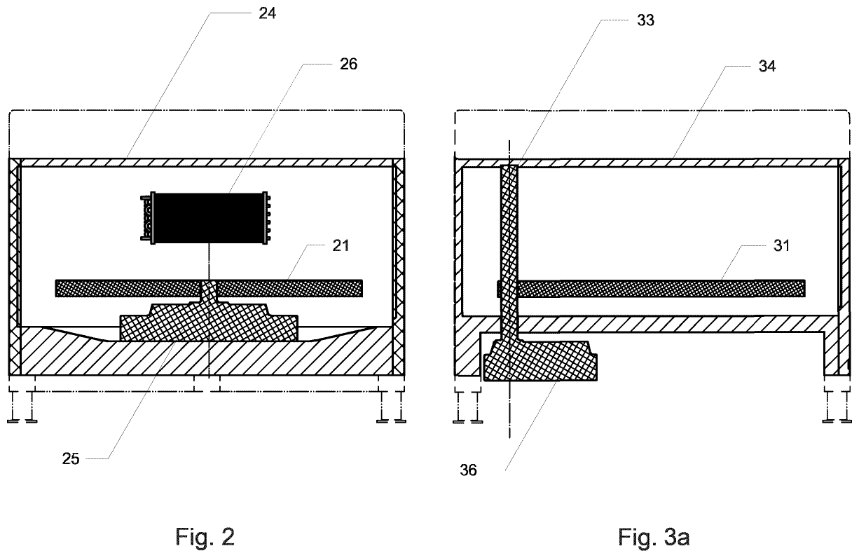

FIG. 2 shows a schematic sectional view of a shaker according to the state of the art. A tray 21 is driven by a motor 25, which is located inside a housing 24. The arrangement of the motor 25 in the interior of the housing 24 has the disadvantage that heat generated by the motor 25 directly heats up the interior and thus the samples located therein. For some samples, however, it is necessary to air-condition the interior, in particular to control the temperature and/or humidity, for example via an air conditioning control 26. As described above, this can lead to condensation of moisture in the interior, in particular on the air conditioning control 26 or the cooler, which in turn can damage the samples. An internal motor 25 contributes to this problem, as the heat generated by the motor 25 must be removed from the interior by the air conditioning control 26.

FIGS. 3a and 3b illustrate a further aspect of the invention with a schematic section through one embodiment of the shaker in each case. In contrast to FIG. 2 (prior art), the tray 31 can be driven here via a main drive shaft 33 by a motor 36, which is mounted outside the housing 34. The main drive shaft 33 runs orthogonally to the tray 31 and for the most part inside the housing 34, while a smaller part of the main drive shaft 33 runs outside the housing. The positioning of the motor 36 below the housing 34 is advantageous with regard to a low center of gravity of the device.

FIG. 3b shows a section through an embodiment of the device, in which the carrier 32 is bearing-mounted via a bearing 36a on a carrier element 35, which is attached to the housing 34. As in FIG. 1c, this achieves a mechanical decoupling of the bearing/mounting of the carrier from the drive, in particular from the main drive shaft 33.

A motor mounted outside the housing, particularly as in FIG. 3a, generally has the advantage that the heat generated by the motor is not introduced into the interior of the housing and therefore does not heat it up. This means that less cooling power is required to keep the interior at a constant temperature. As a result, less moisture condenses in the interior, e.g. locally on the climate control unit or the radiator, which could be harmful to the samples. This makes it easier to create controlled environmental conditions in the interior, in particular a constant temperature and high humidity, e.g. between 80% and 100% relative humidity.

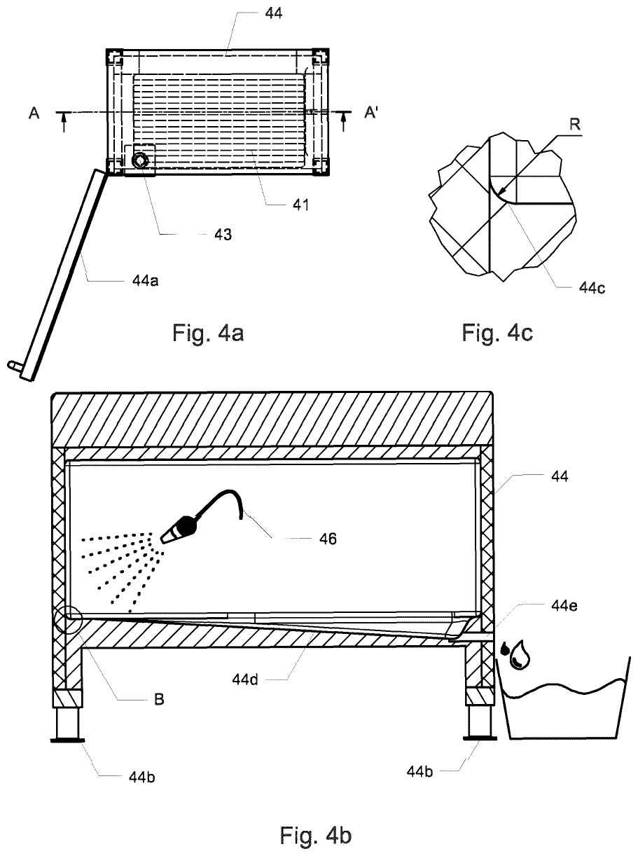

FIGS. 4a to 4c show a further aspect of the invention which improves cleanability by means of a top view of the device (FIG. 4a), a sectional view of the device (FIG. 4b, tray and main drive shaft not shown) and a detailed view of a corner area of the interior (FIG. 4c).

In the top view of FIG. 4a, a housing 44 comprising a door 44a is shown in the open state. In addition, the tray 41 and the main drive shaft 43 are indicated by dashed lines.

FIG. 4b now illustrates the vertical section along the line AA′ from FIG. 4a. The housing 44 comprises feet 44b arranged to support the housing 44. The feet 44b define a bearing surface, in particular as a plane through the feet 44b. With the feet 44b or the bearing surface, the device can be placed on a surface, e.g. a table.

The interior of the housing 44 comprises rounded corners and edges 44c, see the corner area B in FIG. 4b and its detailed view in FIG. 4c. It is advantageous if at least a majority of the corners and edges 44c of the interior are rounded. “Rounded” means in particular that a rounding radius R of the corners and edges 44c is at least 1 mm. Advantageously, the rounding radius R is at least 10 mm, e.g. 15 mm. This prevents sample material or dirt from accumulating in the corners and edges, which would be difficult to clean. For example, the interior can be sufficiently cleaned by spraying, e.g. with the spray nozzle 46.

On the other hand, the underside 44d of the interior can also be inclined, i.e. run at an angle to the bearing surface of the housing. As a result, liquid collects in the interior at the lowest point of the underside 44d, i.e. closest to the bearing surface. An outlet opening 44e is located there through the housing 44, through which the liquid can flow out. This also improves the cleanability of the device, e.g. through the possibility of simply spraying out the interior.

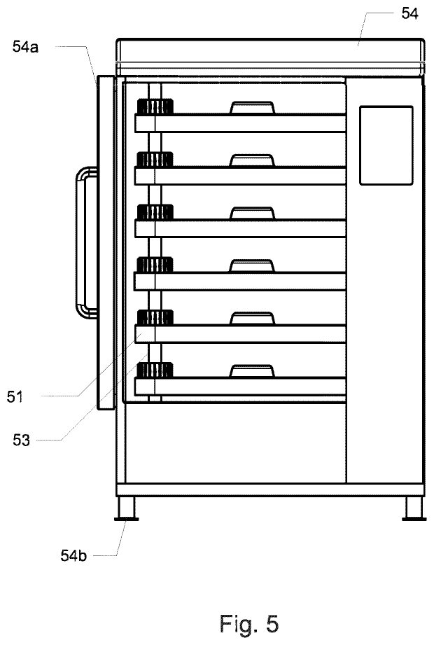

FIG. 5 illustrates a shaker with several, in particular six, trays 51 which are drivable in a housing 54 with door 54a by a single main drive shaft 53. Thus, all trays 51 are drivable by a motor (not shown in FIG. 5), which in turn is mounted outside the possibly air-conditioned interior of the housing 54, as described above. Advantageously, the main drive shaft 53 again extends through the trays 51 in the edge region, in particular in the corner region, for optimum pivoting of the trays 51.

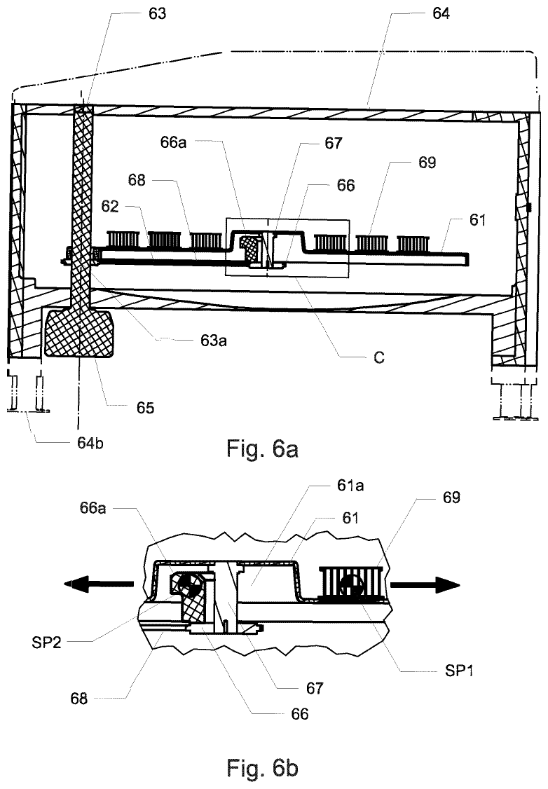

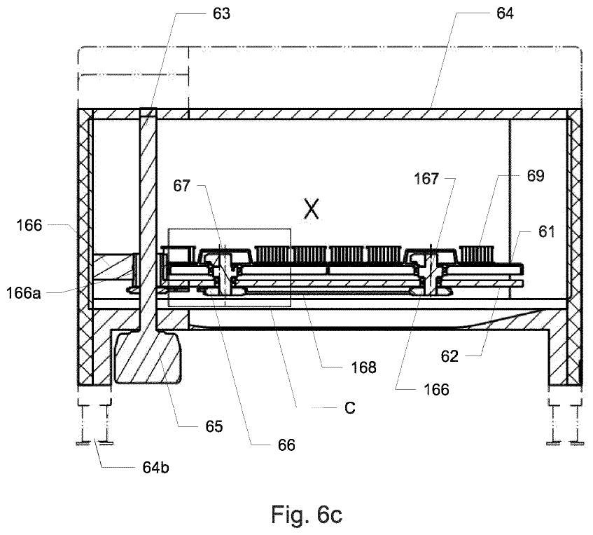

FIGS. 6a and 6b focus on the mechanical aspect of how a tray 61 is pivotably attached to the main drive shaft 63 via a carrier 62 with a bearing 63a (FIG. 6a), as well as details of the drive of the tray 61 via a drive element 66 (FIG. 6b). FIG. 6c shows an alternative solution for mounting the tray 61 via a carrier 62 with a bearing 166a, in particular a plain bearing, on a carrier element 166, which is part of the housing 64 or attached to it. In principle, the mechanisms described can also be applied to several trays, e.g. to the shaker according to FIG. 5.

The tray 61 is set up to be loaded with one or more samples 69, for example in microtiter plates, which are to be shaken. For this purpose, the tray 61 preferably has fastening elements, e.g. for a vessel stand, in order to hold the samples 69 or vessels, in particular microtiter plates, stationary relative to the tray 61 during the shaking process.

The tray 61 is rotatably bearing-mounted on the drive element 66 via a fixed tray shaft 67. The drive element 66 is in turn rotatably attached to the carrier 62, which is pivotably bearing-mounted on the main drive shaft 63 via the bearing 63a. The bearing of the tray shaft 67 in or on the drive element 66 is eccentric, so the axis of rotation of the tray shaft 67 does not coincide with the axis of rotation of the drive element 66. This eccentricity of the tray shaft 67 results in a circular movement when the drive element 66 rotates, on which the tray 61 rotates, and thus the desired shocking of the tray 61 together with the samples 69.

Optionally, the tray 61 is enclosed by a housing 64 when swiveled in, which serves as splash protection and/or for air conditioning the samples as described above. The main drive shaft 63 extends, in particular vertically, i.e. in the direction of gravity, through the housing 64 and is freely rotatably bearing-mounted on it. Furthermore, a motor 65 for driving the main drive shaft 63 is attached, preferably externally, to the housing 64.

In addition, the housing 64 (as already described with reference to FIG. 1a) may comprise feet 64b adapted to support the weight of the device. Generally, the feet may also be adapted for attachment to a support, such as a laboratory bench.

FIG. 6b is an enlarged view of section C in FIGS. 6a and 6c. The tray 61, which can be loaded with samples 69, for example in a microtiter plate, is rotatably bearing-mounted on the drive element 66 via the tray shaft 67. The drive element 66 preferably comprises a pulley which is driven by the main drive shaft via a belt 68. For this purpose, a second pulley is attached to the main drive shaft and the belt 68 is tensioned over the two pulleys.

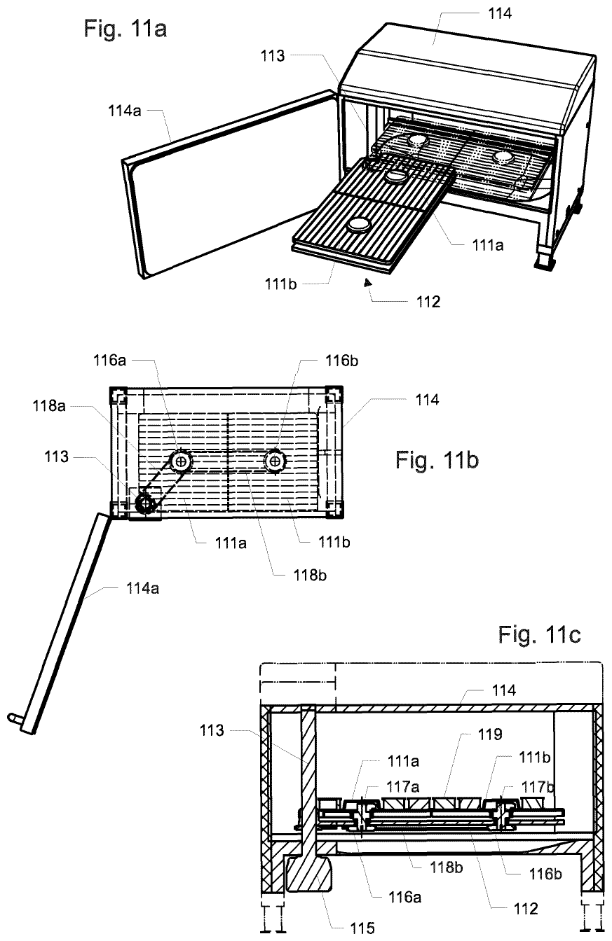

Advantageously, the tray 61 is also eccentrically bearing-mounted on the second pulley in the same way as on the first pulley on the drive element 66. This provides an anti-rotation lock for the tray 61, since its freedom of movement is thus restricted to a circular translation. In addition, as described above, the anti-rotation tray can comprise two tray parts which are connected to each other, for example by a linear guide. Such an embodiment is shown in FIGS. 11a, 11b and 11c, see below.

FIG. 6c also shows-independently of the different mounting of the carrier to FIG. 6a—an advantageous design of the mounting of the tray 61 on the carrier 62. In addition to the drive element 66 and the tray shaft 67, the device comprises a second drive element 166 and a second tray shaft 167. The tray 61 is thus connected both to the tray shaft 67 and to the second tray shaft 167, which are bearing-mounted eccentrically-namely with the same eccentricity-on the drive elements 66 and 166 respectively. To ensure that the two drive elements 66 and 166 rotate synchronously, they are mechanically coupled to one another, for example via a toothed belt 168. This mounting of the tray 61 on the carrier 62 prevents undesired rotation of the tray 61 during the desired orbital movement during shaking.

In general, it is advantageous to compensate for imbalances that occur on rotating components of the device. In addition to the main drive shaft, cf. the section “Multiple trays” above, this applies above all to the drive element 66. Particularly at high shaking frequencies and therefore speeds, e.g. of over 1000 rpm, over 1500 rpm, over 2000 rpm or over 2500 rpm, e.g. with a 3 mm diameter of circular movement, as the device can achieve, an imbalance otherwise leads to vibrations, to increased wear of the bearings and mountings and to excessive noise generation.

FIG. 6b shows an arrangement of a counterweight 66a on the drive element 66, which particularly simply and effectively compensates for an imbalance caused by the eccentric mounting of the tray 61 (with samples 69) on the drive element 66. It is advantageous that the center of gravity SP2 of the counterweight 66a is located in the same plane orthogonal to the axis of rotation of the drive element 66 as the center of gravity SP1 of the tray 61 together with the intended load of samples 69. According to FIG. 6b, this can be solved in such a way that the tray 61 comprises an upward protrusion 61a, under which at least a part of the counterweight 66a is located. The torques exerted by SP1 and SP2 when the drive element 66 rotates about the axis of rotation should generally just cancel each other out. With the arrangement shown, both a static and a dynamic imbalance can be compensated. This makes it possible to achieve high shaking frequencies of over 1000 rpm, in particular over 1500 rpm, over 2000 rpm or over 2500 rpm, with a circular movement diameter of 3 mm, for example, with a space-saving design.

In the embodiment of FIG. 6c, counterweights are also advantageously arranged on the drive elements 66 and 166 for the same reasons. These counterweights are again (analogous to the above description) adapted in such a way that they compensate for both a static and a dynamic imbalance during the orbital movement of the tray 61.

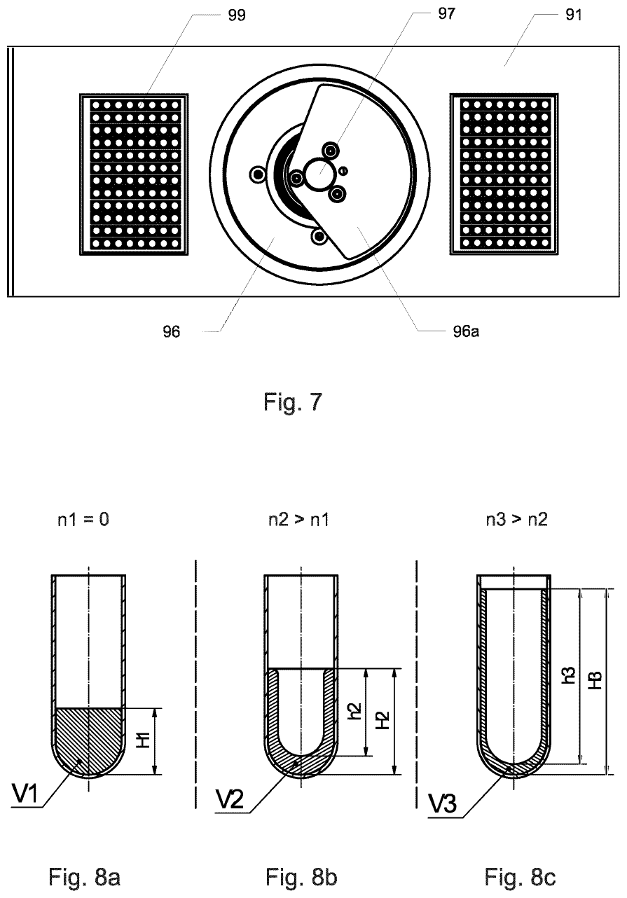

FIG. 7 shows a top view of or a horizontal section through a tray 91 with counterweight 96a. Two microtiter plates with a plurality of samples 99 are mounted on the tray 91, for example by means of vessel holders. As described above in connection with FIG. 6b, the counterweight 96a is attached to the drive element 96, so that an imbalance caused by the eccentric mounting of the tray 91 (with samples 99) on the drive element 96 is compensated particularly simply and effectively. As shown, the counterweight 96a can be attached to the drive element 96, for example with screws. Again, the center of gravity of the counterweight 96a is located in the same plane orthogonal to the axis of rotation of the drive element 96 as the center of gravity of the tray 91 together with the intended load of samples 99.

Advantageously, the counterweight 96a comprises an opening through which the tray shaft 97 extends. In addition, the counterweight 96a can advantageously be shaped similar to a sector of a circle when viewed from above. Both embodiments enable the largest possible volume and thus the largest possible mass of the counterweight 96a, whereby the counterweight 96a can nevertheless rotate with the drive element 96 in the protrusion of the tray 91. This maximizes the space available for the samples 99 on the tray 91.

FIGS. 8a, 8b and 8c illustrate the effect of different shaking frequencies n1, n2 and n3 on a liquid sample in a vessel, e.g. a test tube or a microtiter plate. The sample volume Vis the same in each of the illustrations: V1=V2=V3.

In FIG. 8a, the sample is at rest, i.e. shaking frequency n1=0. The sample liquid has an approximately flat and horizontal surface. The sample liquid fills the vessel to a height H1. This height can be taken as a measure of the diffusion distance d1 that oxygen must travel from the surrounding gas into the sample: d1=H1.

In FIG. 8b, the sample is shaken at a shaking frequency n2>0, e.g. at n2=1000 rpm. The liquid is pressed upwards at the edge of the vessel and a meniscus, i.e. a concave surface of the sample liquid, is formed. While the surface area increases compared to the situation in FIG. 8a, the diffusion distance d2=H2−h2 decreases: d2<d1. Both result in oxygen entering the sample more quickly.

In FIG. 8c, the sample is shaken much faster, n3>n2, e.g. with n3=2000 rpm. The sample liquid is “pulled” far up the edge of the vessel. The surface area increases further as a result of the increasing meniscus and the diffusion distance d3=H3−h3 decreases further: d3<d2. Oxygen transport into the sample is therefore further improved.

This demonstrates one of the major advantages of the device described, which can achieve shaking frequencies of over 1000 rpm, in particular over 1500 rpm, over 2000 rpm or over 2500 rpm: The oxygen transport from the gas phase into the liquid phase, i.e. into the sample, is greatly increased. In particular, cells can be cultivated with a similar rapid growth rate and produce a similar amount of biomass as when cultivated in a bioreactor. Such a device can therefore be used to carry out initial tests during cell cultivation under conditions similar to those later used in the mature process, particularly in the bioreactor.

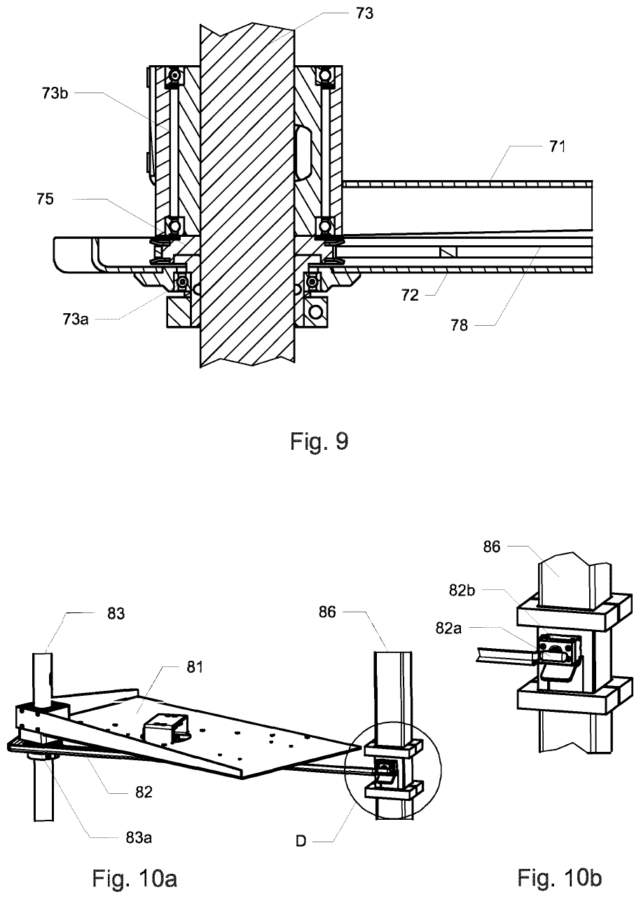

FIG. 9 shows how a tray 71 can be bearing-mounted on a main drive shaft 73 via a carrier 72. Such a mounting is compatible, for example, with the embodiments of FIGS. 1a/b, 3, 5 and 6a/b. As in FIGS. 6a/b, the tray 71 is bearing-mounted eccentrically on a drive element (not shown) with a first pulley. The drive element or the first pulley is rotatably bearing-mounted on the support 72 and is arranged to be driven via the belt 78. The belt 78 also runs over the second pulley 75, which is attached to the main drive shaft 73 and is accordingly driven by the drive or motor via the main drive shaft 73.

The carrier 72, which is designed to support the weight of the tray 71 together with the load of samples, is bearing-mounted on the main drive shaft 73 via a bearing 73a, e.g. a ball bearing. In this way, the support 72 can remain stationary, for example by being locked via a latch as in FIGS. 10a/b, while the main drive shaft 73 rotates. Preferably, the bearing 73a is located below the second pulley 75.

Optionally, the carrier 72 together with the tray 71 can alternatively or additionally also be bearing-mounted above the second pulley 75 on the main drive shaft 73 via a bearing 73b. In general, care must be taken to ensure that the tray has sufficient clearance for its circular translation, which is caused by the eccentric bearing. In particular, an opening in the tray 71 through which the main drive shaft 73 passes in a preferred embodiment must be larger than the diameter of the main drive shaft 73 or than the second bearing 73b, if present, by at least the eccentricity of the bearing.

With a bearing 73a or 73b, several carriers can also be pivotably bearing-mounted one above the other on a main drive shaft 73 and driven simultaneously.

FIGS. 10a and 10b illustrate a possibility of locking a tray 81, which (as described, for example, in connection with FIGS. 6a/b and 9) is attached to a carrier 82 via a drive element (not visible), for the shaking process in the housing or on a support structure 86 in the housing, so that it is temporarily not pivotable about the main drive axis. The support structure 86 can be part of the housing or a separate component that is attached to the housing. For the pivoting of the tray 81, the support 82 is in turn pivotably bearing-mounted on the main drive shaft 83 via a bearing 83a, see e.g. FIG. 9.

FIG. 10b shows the section D of FIG. 10a enlarged. This comprises a first locking element 82a as a latch at or near its end remote from the main drive shaft. As a counterpart to the first locking element 82a, a second locking element 82b is attached to the support structure 86. The first and second locking elements 82a and 82b are in particular designed to establish a detachable connection upon contact. As a result, the carrier 82 can be connected to the support structure 86 for the shaking process and, in particular, pivoting of the carrier 82 about the main drive shaft 83 can be prevented. In addition, part of the weight of the carrier 82 and tray 81 together with the samples can be borne by the support structure 86, which reduces the load on the bearing 83a on the main drive shaft.

In general, the latching means comprises, for example, mechanical or magnetic components for releasably connecting the carrier 82 to the support structure 86. For example, the latching elements 82a and 82b may comprise magnets which are adapted to latch the carrier 82 to the support structure 86 by their mutual attraction. Alternatively, the locking elements 82a and 82b can be designed as a snap lock or as a hinged lock, in which a detachable connection is produced mechanically.

FIGS. 11a, 11b and 11c illustrate an embodiment of the shaker with a divided tray, whereby a particularly simple and reliable anti-rotation device for the tray can be achieved. FIG. 11a is a perspective view analogous to FIG. 1a; FIG. 11b is a schematic section through the shaker in the plane of the drive elements analogous to FIG. 1b; FIG. 11c is a schematic vertical section analogous to FIG. 6a. The features described for the previous embodiments are analogously applicable here.

The shaker comprises a housing 114 with a door 114a, which is arranged to open and close a front side of the housing. In FIGS. 11a and 11b, the door 114a is shown in the open state. Furthermore, the shaker comprises a main drive shaft 113, which can be driven by a motor 115. A carrier 112 is rotatably bearing-mounted on the main drive shaft 113, which can be engaged on the housing, for example by means of a latching mechanism (as described above). In turn, a first drive element 116a and a second drive element 116b are rotatably bearing-mounted on the carrier 112. The first drive element 116a is coupled to the main drive axle 113 via a first belt 118a and is driven by the latter. The second drive element 116b is coupled to the first drive element 116a via a second belt 118b and is thus also driven. It is important that the two drive elements 116a and 116b run synchronously. For this reason, a toothed belt is advantageously used at least for the second belt 118b.

A first tray shaft 117a or a second tray shaft 117b is eccentrically bearing-mounted on or in the first drive element 116a or the second drive element 116b. A first tray part 111a or a second tray part 111b is in turn attached to the first tray shaft 117a or to the second tray shaft 117b, in particular in a non-rotatable manner. Samples 119 can be placed on the tray parts 111a and 111b as described above, for example in test tubes or microtiter plates.

A particularly simple and reliable anti-rotation device for the two tray parts 111a and 111b can now be achieved by means of a flexible connection between the two tray parts (not shown in FIGS. 11a-c). Advantageously, this connection comprises a linear guide between the first tray part 111a and the second tray part 111b. The linear guide may, for example, be fixedly attached to one tray part while allowing the other tray part to slide along the guide. Such a flexible connection avoids damaging forces on the bearings of the tray shafts and drive elements, e.g. as a result of thermal expansion, in particular of the support 112.

While preferred embodiments of the invention are described in the present application, it should be clearly noted that the invention is not limited thereto and may be practiced in other ways within the scope of the following claims.

1. A device for shaking samples comprising
one carrier,
a drive element, which is rotatably bearing-mounted on the carrier,
a tray arranged for loading with the samples,
a tray shaft, which is connected to the tray and is bearing-mounted eccentrically on the drive element,
an openable housing, wherein the tray, the tray shaft, the drive element and the carrier are located in an interior space within the housing,
a main drive shaft drivable by a drive, which extends orthogonally to the tray at least partially through the housing,
wherein the drive element is drivable via the main drive shaft,
wherein the carrier together with the tray, in particular with a bearing, is pivotably bearing-mounted about the main drive shaft,
wherein the carrier together with the tray is at least partially pivotable out of the housing in the open state.

2. The device according to claim 1,
wherein the carrier together with the tray is pivotable out of the housing to more than 50% of a surface of the tray in the open state.

3. The device according to claim 1,
wherein the carrier together with the tray is pivotable through at least 45°, in particular at least 90°, about the main drive shaft.

4. The device according to claim 1,
wherein the tray comprises an opening through which the main drive shaft passes,
wherein the opening is located in an edge region of the tray, in particular less than 20% of a length and/or width of the tray away from an edge of the tray.

5. The device according to claim 1,
wherein the main drive shaft extends outside the surface of the tray.

6. The device according to claim 1,
wherein the carrier comprises a latch at the end remote from the main drive shaft for releasably securing the carrier to the housing or to a support structure in the housing.

7. The device according to claim 1, further comprising
a belt arranged to drive the drive element via the main drive shaft.

8. The device according to claim 1,
wherein the tray shaft is centrally attached to the tray.

9. The device according to claim 1,
wherein the tray is secured against rotation relative to the carrier.

10. The device according to claim 9,
wherein the tray comprises a first tray part and a second tray part,
wherein the first tray part and the second tray part are bearing-mounted eccentrically on a first drive element and a second drive element via a first tray shaft and a second tray shaft, respectively, which are connected to the first and second tray parts, respectively,
wherein the first drive element and the second drive element are rotatably bearing-mounted on the carrier and can be driven via the main drive shaft,
wherein the first and second tray sections are connected to each other by a flexible connection, in particular by a linear guide.

11. The device according to claim 1, further comprising
at least one further tray, in particular at least five further trays, together with a further tray shaft, a further drive element and a further carrier in the interior of the housing, wherein the at least one further drive is drivable via the main drive shaft.

12. The device according to claim 11,
wherein the at least one further tray is pivotably bearing-mounted about the main drive shaft independently of the other tray or trays.

13. The device according to claim 11,
wherein an angular position of a bearing of the further tray shaft on the further drive element deviates from an angular position of the bearing of the tray shaft on the drive element,
in particular where the angular position of the bearings of the various tray shafts differ from one another by 360°/N, where N is the number of trays in the device.

14. The device according to claim 1,
further comprising a climate control element for controlling the temperature and/or humidity in the interior of the housing.

15. The device according to one claim 1,
wherein the drive comprises a motor which is mounted outside the housing, in particular on an underside of the housing,
in particular wherein the main drive shaft extends through an opening in the underside of the housing, and/or
in particular wherein the interior of the housing is thermally decoupled from the motor.

16. The device according to claim 1,
wherein an eccentricity of the bearing of the tray shaft on the drive element is between 0.5 and 50 mm, in particular between 1 and 3 mm.

17. The device according to claim 1,
wherein a shaking frequency of the tray as a result of the drive is at least 1000 rpm, in particular at least 1500 rpm, at least 2000 rpm or at least 2500 rpm.

18. The device according to claim 1,
wherein the tray has a rectangular shape,
in particular wherein the tray has a length of between 50 and 100 cm, and/or
in particular wherein the tray has a width of between 30 and 70 cm.

19. The device according to claim 1,
wherein a part of the housing facing the interior is at least 50% covered with stainless steel.

20. The device according to claim 1,
wherein the part of the housing facing the interior comprises rounded corners and edges,
in particular wherein a radius (R) of the corners and edges is at least 10 mm.

21. The device according to claim 1,
wherein at least a part, in particular at least 70%, of an underside of the interior extends inclined relative to a bearing surface of the housing,
in particular wherein an angle between the underside of the interior and the bearing surface of the housing is between 1° and 30°, in particular between 5° and 15°.

22. The device according to claim 21,
wherein the housing comprises an outlet opening, in particular through the housing, in the region of the underside of the interior closest to the bearing surface, in particular wherein the outlet opening closable.

23. The device according to claim 1, further comprising
a carrier element attached to the housing,
wherein the carrier together with the tray is pivotably bearing-mounted on the carrier element via the bearing, in particular via a plain bearing,
in particular wherein a pivot axis of the carrier coincides with the main drive shaft.
